(19) United States
(12) Patent Application Publication
(10) Pub. No.: US 2026/0315394 A1
(43) Pub. Date: Oct. 15, 2026

(54) NOVEL METHODS FOR PREPARING SEMI-SOLID COMPOSITIONS AND USES THEREOF

(71) Applicant: Hoffmann-La Roche Inc., Little Falls, NJ (US)

(72) Inventors: Peter KÜHL, Lörrach (DE); Si Ying LAI, Basel (CH); Leonie Flurina Claude WAGNER-HATTLER, Allschwil (CH)

(73) Assignee: Hoffmann-La Roche Inc., Little Falls, NJ (US)

(21) Appl. No.: 19/549,309

(22) Filed: Feb. 25, 2026

Related U.S. Application Data
(63) Continuation of application No. PCT/EP2024/074239, filed on Aug. 30, 2024.

(30) Foreign Application Priority Data
Sep. 1, 2023 (EP) 23194881.1

(51) Int. Cl. A61K 9/20; A61K 31/167; A61K 31/192

(52) U.S. Cl. CPC A61K 9/205; A61K 9/2009; A61K 9/2013; A61K 9/2018; A61K 9/2027; A61K 9/2031; A61K 9/2054; A61K 9/2095; A61K 31/167; A61K 31/192

(57) ABSTRACT

The present application discloses methods and uses for preparation of a dry solid composition as a precursor for a stable gel body formation upon addition of water. The invention also discloses the compositions obtained from said methods and uses. Said gel bodies can, for example, be used in pharmaceutical compositions to administer drug substances to patients with difficulties to swallow.

### CROSS-REFERENCE TO RELATED APPLICATIONS

This application is a Continuation of International Patent Application No. PCT/EP2024/074239, filed Aug. 30, 2024, which claims benefit of priority to European Patent Application No. 23194881.1, filed Sep. 1, 2023, each of which is incorporated herein by reference in its entirety.

### FIELD OF THE INVENTION

The present invention generally relates to the preparation of semi-solid, gel-forming compositions. More specifically, the present invention relates to the field of semi-solid pharmaceutical dosage forms for oral administration of drugs to patient populations with difficulties to swallow.

### BACKGROUND OF THE INVENTION

Solid formulation that are used for instant preparation of a semi-solid, jelly form for the administration of drug, particularly for those patients who may have difficulties in swallowing solid dosage forms, are known in the art.

WO2005/107713 discloses swellable powder systems forming a gel within 5 minutes without application of mixing forces. The formation of a gel is primarily related to the use of gellan gum that are further combined with swelling and gelling agents. Powder blend can be filled into spoon, which sealed foil. This relates to the original Parvulet system invented by Bar-Shalom.

WO2022/011341 A1 discloses gel forming tablets or mini-tablets that primarily contain gellan gum as gel forming agent and an osmogen. The regular tablets convert to the semi-solid form within 30-90 seconds.

However, there remains a need for alternative approaches, for example, to provide more rapidly gel forming tablets without using gellan gum. The present invention solves this problem by using a combination of alternative gel formers (gelling agents) together with wicking agents.

### BRIEF DESCRIPTION OF THE FIGURES

FIG. 1: Tablet holder with ring.

FIG. 2: Water uptake of tablet using tensiometer with ring holder for different binary combinations of gel formers; tablet weight dry: 350 mg.

FIG. 3: Viscosities in dependency of shear rate for various gelled tablet systems combining two gel formers and a wicking agent, tablet weight: 350 mg

FIG. 4: Water uptake of tablets using tensiometer with ring holder containing kappa-carrageenan, polyethylene oxide and calcium silicate; tablet mass dry: 350 mg.

FIG. 5: Water uptake of tablets using tensiometer with ring holder containing kappa-carrageenan, sodium polyacrylate and calcium silicate, tablet mass dry: 350 mg.

FIG. 6: Drug release from gelled tablet: formulation system with polyethylene oxide and kappa-carrageenan optionally containing 28.4% drug, tablets with 353 mg weight and a solid fraction of 0.75, Ibuprofen in FaSSIF (fasted simulated intestinal fluid; (A)) pH=6.5 and Acetaminophen in SGF (simulated gastric fluid; (B)) pH=2.0.

FIG. 7: Water uptake of gelling tablet containing optionally a drug, formulation system with polyethylene oxide and kappa-carrageenan.

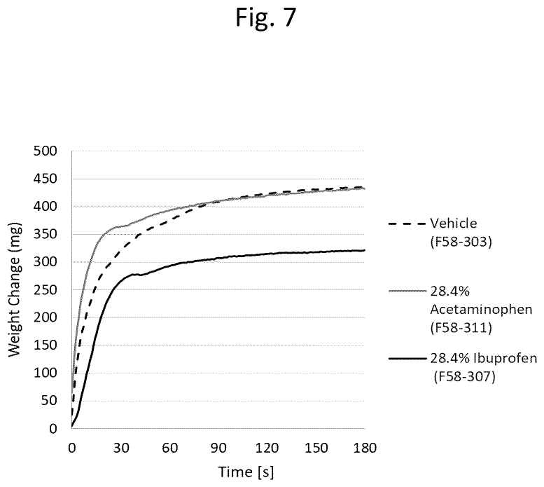

FIG. 8: Gelling behavior of a tablet over time described by images at the example of formulation with 2.5% polyethylene oxide, 2.5% kappa-carrageenan and 5% calcium silicate (F58-2).

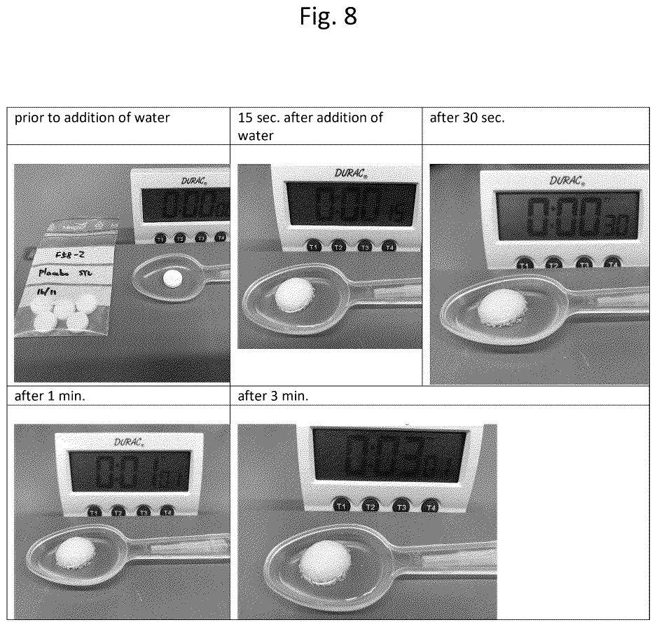

FIG. 9: Gelling behavior of a tablet over time described by images at the example of formulation with 7.5% polyethylene oxide, 7.5% kappa-carrageenan and 10% calcium silicate (F58-10).

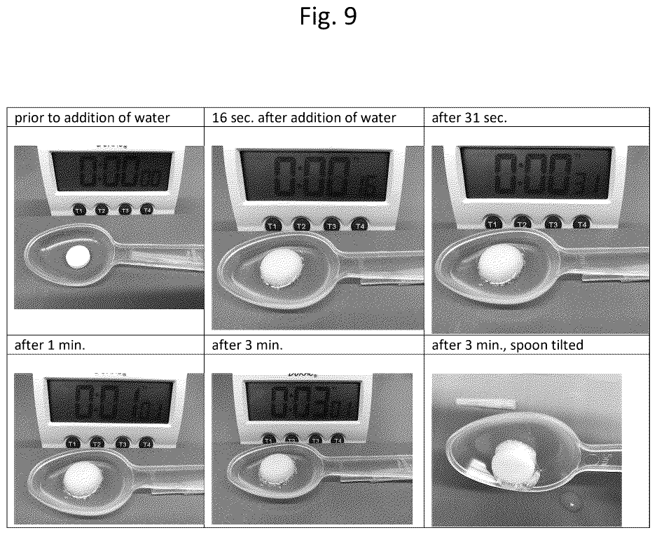

### DETAILED DESCRIPTION OF THE INVENTION

The present invention provides improved dry solid compositions for use in the preparation of semi-solid gel bodies. Said gel bodies are formed upon addition of water to the present dry solid compositions and can, for example, be used as oral dosage form for the administration of drugs to patients with difficulties to swallow.

Technical problems often associated with the preparation of such dosage forms involve the timing of gel formation, the stability and homogeneity of the obtained gel as well as the drug load in—and drug release from such jelly dosage forms.

The present invention provides a solution to these problems by providing novel combinations of gel forming agents and wicking agents and their use in the preparation of dry solid compositions which lead to a rapid gel-formation upon the addition of water. The present compositions and uses also lead to stable, homogenous gels with an improved drug load and a favorable drug-release profile. The term “rapid” as used herein means within 180 seconds; or within 120 seconds; or within 60 seconds; or within 30 seconds. In one embodiment, rapid means within 30 seconds. In another embodiment, rapid means any time between 30 and 180 seconds.

In one embodiment, the present invention provides the combined use of two gel forming agents and a wicking agent in the preparation of a dry solid composition as a precursor for a stable gel body formation upon addition of water to said dry solid composition. In one embodiment, 2 mL of water was added to a dry solid 350 mg tablet on a spoon using a plastic pipette.

The term gel forming agent means an agent independently selected from kappa-carrageenan (Ph.Eur., USP-NF), polyethylene oxide (USP-NF), pectin (USP), sodium polyacrylate (polymerization degree: 2.700-7.500) and sodium alginate (Ph.Eur., USP-NF, 60-170 mPas for 1% solution). In one embodiment, the gel forming agent is polyethylene oxide with an average molecular weight (MW) of about 100.000 g/mol. In yet another embodiment, the following combinations of gel forming agents and respective ratios are preferred:
1. kappa-carrageenan combined with sodium polyacrylate at ratios of 2.5:1 to 75:1 (max. 7.6% in total of the dry tablet mass).
2. kappa-carrageenan combined with polyethylene oxide at ratios of 3:1 to 1:3. In this embodiment the polyethylene oxide preferably has an average molecular weight (MW) of about 100.000 g/mol (max. 10.0% in total of the dry tablet mass).
3. kappa-carrageenan combined with sodium alginate at ratio of 30:1 to 50:1 (max. 5.1% in total of the dry tablet mass)
4. polyethylene oxide combined with pectin at ratio of 1:1 (max. 10.0% in total of the dry tablet mass)
5. polyethylene oxide combined with sodium polyacrylate at ratio of 10:1. (max. 5.5% in total of the dry tablet mass)
6. polyethylene oxide combined with sodium alginate at ratio of 30:1 to 50:1 (max. 7.6% in total of the dry tablet mass)

If not explicitly stated otherwise, ratios used throughout this specification are weight ratios (w/w).

The term wicking agent as used herein means an agent independently selected from highly porous calcium silicate (USP-NF) and highly porous magnesium aluminometasilicate (JP, USP-NF, Ph.Eur). In one embodiment, the wicking agent is highly porous calcium silicate at a concentration of about 5 to about 10% (w/w) in the dry solid composition. In another embodiment, the wicking agent is highly porous magnesium aluminometasilicate at a concentration of about 7 to about 8% (w/w), preferably at about 7.5% (w/w) in the dry solid composition.

In yet another embodiment, the present invention provides any of the uses described hereinbefore for the preparation of a pharmaceutical composition, preferably a dry tablet, which comprises an active pharmaceutical ingredient and, optionally, additional pharmaceutically acceptable excipients.

The term active pharmaceutical ingredient (API) means any therapeutically active agent or a drug, which is suitable for oral administration, preferably by administration via a tablet or a gel. In one embodiment, the API is a small molecule or an oligonucleotide. The terms “small molecule” and “oligonucleotide” are well known to a person of skill in the art, for example a medical practitioner or a medicinal Chemist. A huge number of small molecule APIs is already approved or in development such as, for example, acetoaminophen or ibuprofen which are used herein as model compounds. In one embodiment, the term “small molecule” refers to molecules, whether naturally-occurring or artificially created (e.g., via chemical synthesis) that have a relatively low molecular weight. Typically, a small molecule is an organic compound (i.e., it contains carbon). The small molecule may contain multiple carbon-carbon bonds, stereocenters, and other functional groups (e.g., amines, hydroxyl, carbonyls, and heterocyclic rings, etc.). In certain embodiments, the molecular weight of a small molecule is not more than about 1,000 g/mol, not more than about 900 g/mol, not more than about 800 g/mol, not more than about 700 g/mol, not more than about 600 g/mol, not more than about 500 g/mol, not more than about 400 g/mol, not more than about 300 g/mol, not more than about 200 g/mol, or not more than about 100 g/mol. In certain embodiments, the molecular weight of a small molecule is at least about 100 g/mol, at least about 200 g/mol, at least about 300 g/mol, at least about 400 g/mol, at least about 500 g/mol, at least about 600 g/mol, at least about 700 g/mol, at least about 800 g/mol, or at least about 900 g/mol, or at least about 1,000 g/mol. Combinations of the above ranges (e.g., at least about 200 g/mol and not more than about 500 g/mol) are also possible. In certain embodiments, the small molecule is a therapeutically active agent such as a drug (e.g., a molecule approved by the U.S. Food and Drug Administration as provided in the Code of Federal Regulations (C.F.R.)). The small molecule may also be complexed with one or more metal atoms and/or metal ions. In this instance, the small molecule is also referred to as a “small organometallic molecule.” Preferred small molecules are biologically active in that they produce a biological effect in animals, preferably mammals, more preferably humans. In certain embodiments, the small molecule is a drug. Preferably, though not necessarily, the drug is one that has already been deemed safe and effective for use in humans or animals by the appropriate governmental agency or regulatory body. For example, drugs approved for human use are listed by the FDA under 21 C.F.R. §§ 330.5, 331 through 361, and 440 through 460, incorporated herein by reference; drugs for veterinary use are listed by the FDA under 21 C.F.R. §§ 500 through 589, incorporated herein by reference. In another embodiment the API is a small molecule drug suitable for oral administration to a patient, preferably a patient with difficulties to swallow.

Pharmaceutically acceptable excipients are known to a person of ordinary skill in preparing/providing pharmaceutical dosage forms such as tablets. In one aspect in accordance with the present invention, pharmaceutically acceptable excipients are those typically used to prepare oral dosage forms. In another aspect pharmaceutically acceptable excipients may be mannitol or microcrystalline cellulose (see e.g., Table 2). Patients with difficulties to swallow may, for example be pediatric or geriatric patients or patients with certain types of diseases which do not allow them to swallow easily.

In one embodiment, the API is present in the pharmaceutical compositions, obtained from the uses in accordance with the present invention, in an amount up to 30% (w/w) of the final drug product; or in an amount ranging from 25-30% (w/w) of the final drug product.

In yet another embodiment, the present invention provides the uses as defined herein before for the preparation of a stable gel body upon the addition of water to said dry solid composition or tablet. In a preferred embodiment, said stable gel body is formed within 30 seconds after the addition of water.

The term stable gel body as used herein means a gel body which shows sufficient gel firmness, no disintegration into fractional parts and no solid dry core after a maximum of 3 minutes subsequent to the addition of water.

The term FaSSIF has its ordinary meaning known to a person of skill in the art. In one embodiment, FaSSIF is as for example described in S. Klein, The Use of Biorelevant Dissolution Media to Forecast the In Vivo Performance of a Drug, AAPS Journal, Vol. 12, No. 3, September 2010 (#2010) DOI: 10.1208/s12248-010-9203-3.

The term SGF (simulated gastric fluid) means 0.01 N HCl with 2 g/L sodium chloride, pH adjusted with 1 N HCl.

In yet another embodiment the present invention provides the uses as described herein before for the preparation of an oral dosage form for the administration of medicaments to patients with difficulties to swallow such as, for example, pediatric or geriatric patients or patients whose disease state does not allow them to swallow easily.

In still another embodiment the present invention provides a dry solid composition, stable gel body or pharmaceutical composition obtained through any of the uses as defined herein before.

The following table (Table 1) summarizes parameters and criteria to describe targeted properties of compositions (dry solid compositions and gels) in accordance with the present invention.

TABLE 1
Features and related parameters describing properties of suitable gel forming tablets

| Feature | Parameter | Test Instrument | Criteria |
|---|---|---|---|
| Rapid water uptake | Mass increase after 30 | Tensiometer | At least 50% related |
| and no | seconds | equipped with ring | to amount after 3 min. |
| disintegration | | holder | after 30 s |
| Rapid progression | Indentation depth | Texture analyzer | At least 50% related |
| of gelling | in the gelling tablet | | to thickness of gelled |
| | after 30 seconds | | tablet after 30 s |
| | exposure to water | | |
| | Work of penetration | Texture analyzer | Ranges as described |
| | after 30 seconds | | |
| | exposure to water | | |
| Semi-sold property | Viscosity at a shear | Rheometer | >5 Pa*s, more |
| of gelled tablets | ate of 50 1/s | | favorable >10 Pa*s |
| | Stress at flow point | Rheometer | Less than 20 kPa |

The following table (Table 2) summarizes Materials (Ingredients) used to make compositions in accordance with the present invention.

TABLE 2
Materials used in the present composition

| Ingredient - | | | | |
|---|---|---|---|---|
| Generic name | Acronym | Brand Name | Supplier | Function |
| Polyethylene oxide | PO | POLYOX ™ WSR | Dupont Inc., US | Gel |
| | | N10 | | former |
| Kappa carrageenan | CA | Gelcarin ® PH-911 | FMC Biopolymer, US | Gel |
| | | | | former |
| Pectin | PE | VIVAPHARM ® | JRS Pharma, US | Gel |
| | | Pectin USP | | former |
| Sodium | SP | Sodium | FUJIFILM Wako | Gel |
| polyacrylate | | polyacrylate | Pure, Chemical | former |
| | | | Corporation, | |
| | | | Japan | |
| Sodium alginate | SA | Manucol ® KLX | Dupont Inc., US | Gel |
| | | | | Former |
| Magnesium | NS | Neusilin ® US2 | Fuji Chemical | Wicking |
| Aluminum Silicate | | | Industries | agent |
| | | | Co., Ltd., Japan | |
| Calcium silicate | FR | FLORITE ® R | Tomita | Wicking |
| | | | Pharmaceutical | agent |
| | | | Co., Ltd., Japan | |
| Mannitol | (MAN) | PEARLITOL ® 160 | Roquette Frères, | Filler |
| | | C | France | |
| Microcrystalline | (MCC) | CEOLUS ™ PH | Asahi Kasei, Japan | Filler |
| cellulose | | 101 | | |
| Sodium stearyl | | PRUV ® | JRS, Switzerland | Lubricant |
| fumarate | | | | |

### Methods of Manufacture

The amounts of ingredients are weighed, subsequently mixed in a tumble bin mixer for a time equivalent to approximately 160 rotations. The mixed blend is then sieved with a 1 mm meshed sieve followed again by mixing in a tumble bin mixer at same time and speed. The amount of lubricant is sieved and added to the blend and mixed. Optionally, the final dry blend is dry granulated using a roller compactor at a compaction force of approximately 3 kN/cm. The granules are screen milled with approx. 1 mm meshed screen. Finally, the amount of lubricant is sieved and added to the blend or granules and mixed for another time equivalent to approx. 50 to 160 rotations. The final blends are compressed to tablets using a single punch or rotary press at targeted solid fraction that results in sufficient tablet hardness and acceptable friability, e.g., 0.75 to 0.80. For placing the tablets on a teaspoon the tablet are flat-faced round shaped with a diameter of 11 mm and tablet weight 350 mg nominal, and 352 mg for lubricated final blends retrospectively.

The tablets can be placed on a teaspoon or medical spoon with an approximate volume capacity of 5 ml and the 2 mL tap water is added to the spoon. The incubation time of the water for swelling and gelling is not more than 3 min until an administration is appropriately instructed.

The following Tables 3-5 summarize tablet compositions, and corresponding codes, prepared and tested:

TABLE 3
Tablet Compositions for suitable combinations
of gel formers, tablets with 350 mg weight

| | PO | CA | SP | PE | NS | FR | MAN | MCC |
|---|---|---|---|---|---|---|---|---|
| F46 | 5% | — | 0.5% | — | 7.5% | — | 62.0% | 25% |
| F47 | 5% | — | — | 5% | 7.5% | — | 57.5% | 25% |
| F48 | 5% | 5% | — | — | 7.5% | — | 57.5% | 25% |
| F50 | — | 5% | 0.5% | — | 7.5% | — | 62.0% | 25% |
| F56 | 5% | — | 0.5% | — | — | 7.5% | 62.0% | 25% |
| F57 | 5% | — | — | 5% | — | 7.5% | 57.5% | 25% |
| F58 | 5% | 5% | — | — | — | 7.5% | 57.5% | 25% |
| F60 | — | 5% | 0.5% | — | — | 7.5% | 62.0% | 25% |

TABLE 4
Tablet Compositions for suitable ranges of gel formers PO
and CA, and wicking agent FR, tablets with 350 mg weight

| Code | PO | CA | FR | MAN | MCC |
|---|---|---|---|---|---|
| F58-01 | 5% | 5% | 7.5% | 57.5% | 25% |
| F58-02 | 2.5% | 2.5% | 5% | 65% | 25% |
| F58-03 | 2.5% | 2.5% | 10% | 60% | 25% |
| F58-04 | 2.5% | 7.5% | 5% | 60% | 25% |
| F58-05 | 2.5% | 7.5% | 10% | 55% | 25% |
| F53-06 | 5% | 5% | 7.5% | 57.5% | 25% |
| F58-07 | 7.5% | 2.5% | 5% | 60% | 25% |
| F58-08 | 7.5% | 2.5% | 10% | 55% | 25% |
| F58-09 | 7.5% | 7.5% | 5% | 55% | 25% |
| F58-10 | 7.5% | 7.5% | 10% | 50% | 25% |
| F58-11 | 5% | 5% | 7.5% | 57.5% | 25% |

TABLE 5
Tablet Compositions for suitable ranges of gel formers SP
and CA, and wicking agent FR, tablets with 350 mg weight

| Code | SP | CA | FR | MAN | MCC |
|---|---|---|---|---|---|
| F60-01 | 0.5% | 5% | 7.5% | 62% | 25% |
| F60-02 | 0.1% | 2.5% | 5% | 67.4% | 25% |
| F60-03 | 0.1% | 2.5% | 10% | 62.4% | 25% |
| F60-04 | 0.1% | 7.5% | 5% | 62.4% | 25% |
| F60-05 | 0.1% | 7.5% | 10% | 57.4% | 25% |
| F60-06 | 0.5% | 5% | 7.5% | 62% | 25% |
| F60-07 | 1% | 2.5% | 5% | 66.5% | 25% |
| F60-08 | 1% | 2.5% | 10% | 61.5% | 25% |
| F60-09 | 1% | 7.5% | 5% | 61.5% | 25% |
| F60-10 | 1% | 7.5% | 10% | 56.5% | 25% |
| F60-11 | 0.5% | 5% | 7.5% | 62% | 25% |

The invention will now be further illustrated by the following, non-limiting working examples

### EXAMPLES

### Methods of Characterization

Different methods were used to describe the properties and performance of the gel forming tablets with respect to the intended use.

### Water Uptake Over Time

The amount of water ingress is detected by KRÜSS Tensiometer K100 Mk2 (Krüss GmbH, Germany) with 80 mL tap water at room temperature (20° C.). The water uptake of the excipients is determined with the Washburn method setup. 100 mg of the polymer powder was poured into a glass tube with a glass filter and carefully tapped for 5 times. The change in weight was recorded and interpreted as the amount of water absorption.

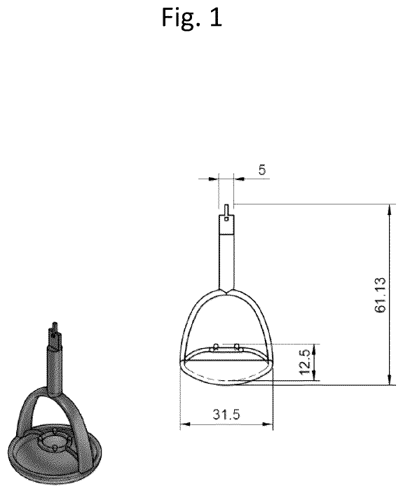

As a holder for the tablets a tailor-made, customized design was applied to the holder in order to reflect possible weight losses caused by a disintegration process during water uptake. The design of the tablet holder including a ring, on which the tablets are placed, is presented in FIG. 1. Tablets with gel-forming properties can show in the test configuration that they have a suitable gel body formed during water uptake, which is demonstrated with the lack of significant weight losses.

### Gelling Performance

The gelling performance of the tablets was characterized using a Texture Analyzer TA-XT2i (Stable Micro Systems, UK). The Texture analyzer is equipped with a 5 kg load cell, a flat-ended cylindrical probe of 7 mm in diameter is used, and the initial probe distance is set to 10 mm. The tablets are swelled with 2 mL of tap water for 30 seconds on a spoon then transferred to a glass slide or directly on a medical spoon for measurement at room temperature (approx. 20° C.). The maximum force is set to 80 g, which is the maximum mouth force of a 2 year-old child, in the “Measure Distance in Compression” mode. The indentation depth and total work of penetration is calculated. The indentation depth is calculated by the percentage of the detected swelled height and the gelling height. The total work of penetration is the integral of the traveled distance and the force applied during the measurement time.

The progression of gelling front is determined by the indentation distance into the tablet from the maximum force of the probe. After 30 sec incubation time in water the indentation depth of gel forming tablets with suitable fast gelling progression, should be at least 50%. The total work of penetration is used describe the range of gel strength for the gel forming tablets identified to meet the targeted properties. The found values for the total work range from 2.47 to 11.04 mJ.

### Rheological Properties

Rheometer HAAKE™ MARS™ II (Thermo Electron GmbH, Germany) is used to determine the rheological properties of the tablets. The tablets were swelled with 2 mL of tap water on a tablespoon for 1 minute then carefully transferred to the measurement plate. A cone plate geometry (20 mm diameter cone with 2°, C20/2) is used, and the measurements were carried out following the regulatory tutorial for rheology proposed by Simoes et al. (Pharmaceutics 12.9 (2020): 820). The results were analyzed with the software HAAKE™ RheoWin™ Data Manager. All measurements were performed at 23° C.

Viscosity at a shear rate of 50 Hz is at minimum 10 Pa*s to obtain a semi-solid gel system. Shear viscosity at 50 Hz should be not less than approximately 10 Pas to avoid potential dose losses due spillages of liquid form during administration of the gelled preparation on the spoon.

Amplitude sweep tests between 0.01 and 600 Pa at 1 Hz were performed to estimate the linear viscoelastic region (LVR) and the flow point is defined as the crossover point of the storage modulus and the loss modulus (G′ and G″). The flow point represents the stress required to transfer the gel into liquid behavior. The stress of the flow point should be lower than the typical strength of tongue that applies stresses ranging from 20 kPa for 2 years old child to 60 kPa for a teenager (see e.g. Potter, Nancy L., and Robert Short; Dysphagia 24.4 (2009): 391-397 and Park, Ji-Su, Dong-Hwan Oh, and Moonyoung Chang, Journal of Physical Therapy Science 28.2 (2016): 442-445.). Therefore, gelled tablets should have flow point stress less than 20 kPa.

### Dissolution

The drug release from the gelled tablets was analyzed with a dissolution tester equipped with inline UV-Vis and HPLC analysis. Dissolution tests were performed with Sotax Dissolution System DS113_AT 7smart (SOTAX AG, Switzerland) according to the USP dissolution II paddle method at a rotation speed of 50 rpm in 900 mL of medium at 37° C. for 1 hour, and an unlimited condition with a rotation speed of 500 rpm for 75 minutes.

### Example 1: Combination of Gel Formers and Wicking Agent

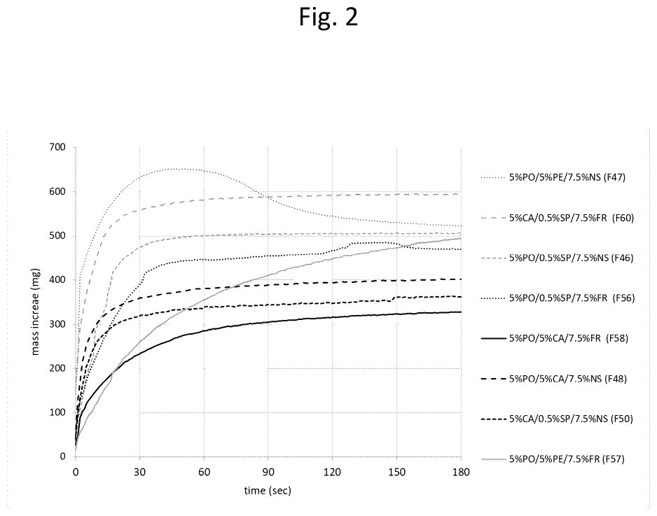

Tablets containing 7.5% (m/m) wicking agents and a combination of 2 gel formers at concentrations as listed in Table 3 resulted in rapid uptake of water and a stable gel body, which does not disintegrate. The results of the water uptake analysis determined with tensiometer using the ring holder show at least more than 50% increase within first 30 seconds and no major weight loss after 180 sec. The combination of 5% PO with 5% Pectin is ideally combined with calcium silicate as wicking agent to obtain most stable gel body (FIG. 2).

The formulations identified show an advanced progression of a gelling as expressed by the results of the texture analyzer. After incubation with water for 30 seconds all formulations showed at least an indentation depth of 50% and the gel firmness at this timepoint is ranging between approx. 1.96 to 9.81 mJ (Table 6).

TABLE 6
Results of the characterization of gel layer after 30 sec with
the Texture Analyzer, average ± SD (n = 3):

| Tablet Variant | Penetration Work (mJ) | Indentation Depth (%) |
|---|---|---|
| 5% PO/0.5% SP/7.5% NS (F46) | 4.24 ± 1.37 | 75.0 ± 1.5 |
| 5% PO/5% PE/7.5% NS (F47) | 2.47 ± 0.49 | 93.7 ± 0.3 |
| 5% PO/5% CA/7.5% NS (F48) | 7.08 ± 1.02 | 85.2 ± 1.2 |
| 5% CA/0.5% SP/7.5% NS (F50) | 10.26 ± 0.72 | 85.4 ± 1.1 |
| 5% PO/0.5% SP/7.5% FR (F56) | 5.33 ± 1.76 | 50.8 ± 16.8 |
| 5% PO/5% PE/7.5% FR (F57) | 5.86 ± 0.98 | 56.6 ± 8.6 |
| 5% PO/5% CA/7.5% FR (F58) | 6.69 ± 1.18 | 76.2 ± 14.9 |
| 5% CA/0.5% SP/7.5% FR (F60) | 8.98 ± 1.60 | 79.1 ± 6.3 |

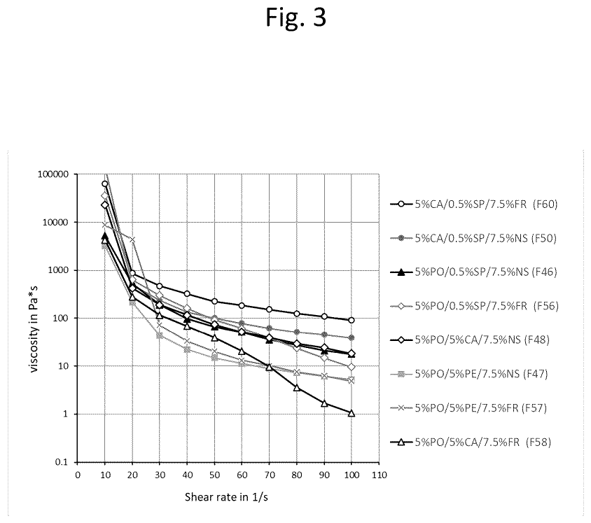

The rheological properties of the fully gelled tablets are shown for the shear stress dependent viscosity in FIG. 3. The gel exhibit a thixotropic shear thinning effect but at a shear rate of 50 l/s the viscosity is still above 10 Pa*s, which shows the semi-solid character of all formulations. The viscosities at a shear rate of 50 l/s and the flow point stresses are summarized in FIG. 3. The flow point stresses are all below 20 kPa indicating the soft character of the gels (Table 7).

TABLE 7
Results of the characterization of rheological gel properties after 3
min with the Haake Rheometer, average ± SD (n = 3):

| Tablet Variant | Flow point (kPa) | Shear viscosity (Pa*s at 50 Hz) |
|---|---|---|
| 5% PO/0.5% SP/7.5% NS (F46) | 0.67 ± 5.35 | 114 ± 46.0 |
| 5% PO/0.5% SP/7.5% FR (F56) | 5.15 ± 0.77 | 74.4 ± 23.3 |
| 5% PO/5% PE/7.5% NS (F47) | 2.29 ± 0.89 | 15.8 ± 3.7 |
| 5% PO/5% PE/7.5% FR (F57) | 5.44 ± 2.35 | 52.8 ± 28.3 |
| 5% CA/5% PO/7.5% NS (F48) | 12.42 ± 3.97 | 68.9 ± 42.0 |
| 5% CA/5% PO/7.5% FR (F58) | 10.44 ± 1.67 | 96.5 ± 62.6 |
| 5% CA/0.5% SP/7.5% NS (F50) | 11.95 ± 2.86 | 123 ± 64.2 |
| 5% CA/0.5% SP/7.5% FR (F60) | 13.49 ± 2.67 | 240.0 ± 7.8 |

### Example 2: Formulations with Carrageenan and Polyethylene Oxide as Gel Formers, and Highly Porous Calcium Silicate as Wicking Agent

The following formulation parameters were used:
2.5 to 7.5% polyethylene oxide
combined with 2.5 to 7.5% kappa-carrageenan, and
5 to 10% highly porous calcium silicate

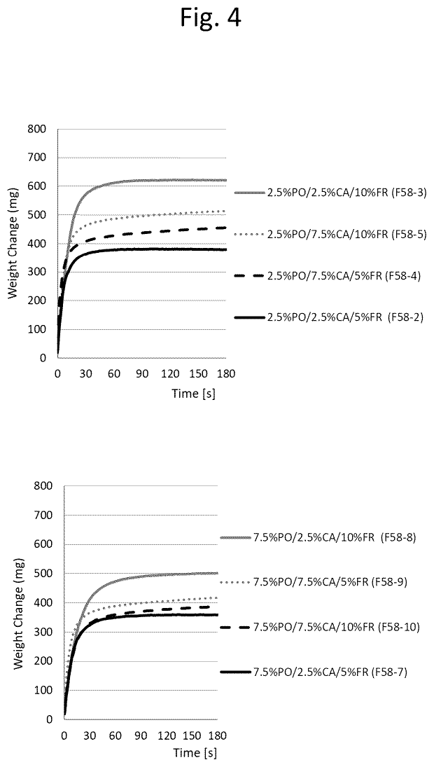

All formulations show rapid water uptake of more than 80% after 30 seconds and no decrease during 180 seconds demonstrating desired absence of disintegration during gelling (FIG. 4).

The indentation depth after 3 seconds is approx. 62% at minimum and the work of penetration ranges from 3.44 to 11.03 mJ. The flow point is for all formulation below 20 kPa and the shear viscosity consistently above 10 Pa*s. The stress at flow point is consistently at 15.2 at maximum and the shear viscosity 35 Pas at minimum (Table 8).

TABLE 8
Results of the characterization of gelling progression after
30 s and rheological properties after 3 min for systems with
polyethylene oxide and kappa-carrageenan, average ± SD (n = 3):

| | | | Rheometer | |
|---|---|---|---|---|
| | Texture analyzer | | | Shear |
| | | Penetration | | viscosity |
| | Indentation | Work | Flow point | (Pa*s at 50 |
| Tablet Variant | depth (%) | (mJ) | (kPa) | 1/s) |
| 2.5% PO/2.5% CA/5% FR | 71.7 ± 2.2 | 10.23 ± 0.32 | 15.2 ± 0.1 | 52.3 ± 36.7 |
| (F58-2) | | | | |
| 2.5% PO/2.5% CA/10% FR | 70.9 ± 1.3 | 4.94 ± 0.23 | 6.7 ± 4.1 | 45.6 ± 18.2 |
| (F58-3) | | | | |
| 2.5% PO/7.5% CA/5% FR | 77.9 ± 1.9 | 3.44 ± 0.09 | 7.4 ± 0.2 | 35.0 ± 13.3 |
| (F58-4) | | | | |
| 2.5% PO/7.5% CA/10% FR | 61.6 ± 3.5 | 7.19 ± 0.32 | 7.8 ± 0.9 | 106.1 ± 13.5 |
| (F58-5) | | | | |
| 7.5% PO/2.5% CA/5% FR | 76.7 ± 6.1 | 5.01 ± 0.95 | 9.1 ± 3.7 | 35.2 ± 19.8 |
| (F58-7) | | | | |
| 7.5% PO/2.5% CA/10% FR | 75.4 ± 1.3 | 8.48 ± 0.31 | 7.9 ± 3.1 | 64.8 ± 61.0 |
| (F58-8) | | | | |
| 7.5% PO/7.5% CA/5% FR | 78.0 ± 15.9 | 11.03 ± 1.29 | 13.8 ± 3.6 | 80.2 ± 20.4 |
| (F58-9) | | | | |
| 7.5% PO/7.5% CA/10% FR | 78.8 ± 2.0 | 8.84 ± 0.97 | 9.8 ± 0.8 | 39.6 ± 40.2 |
| (F58-10) | | | | |
| 5% PO/5% CA/7.5% FR | 78.5 ± 1.5 | 6.44 ± 0.28 | 9.1 ± 0.4 | 42.4 ± 9.3 |
| (F58-1) | | | | |

### Example 3: Formulations with Carrageenan and Sodium Polyacrylate as Gel Formers, and Highly Porous Calcium Silicate as Wicking Agent

The following formulation parameters were used:
0.1 to 0.5% sodium polyacrylate
combined with 2.5 to 7.5% kappa-carrageenan, and
5 to 10% highly porous calcium silicate

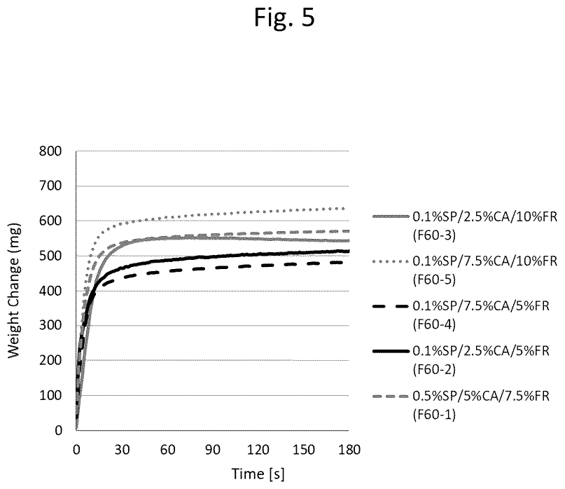

All formulations show rapid water uptake of more than 80% after 30 seconds and no decrease during 180 seconds demonstrating desired absence of disintegration during gelling (FIG. 5).

The indentation depth after 3 seconds is approx. 64% at minimum and the work of penetration ranges from 5.18 to 10.92 mJ. The flow point is for all formulation below 20 kPa and the shear viscosity consistently above 10 Pas (Table 9).

TABLE 9
Results of the characterization of gelling progression after 30
s and rheological properties after 3 min for systems with sodium
polyacrylate and kappa-carrageenan, average ± SD (n = 3):

| | Texture analyzer | | Rheometer | |
|---|---|---|---|---|
| | | Penetration | | Shear viscosity |
| | Indentation | Work | Flow point | (Pa*s at 50 |
| Tablet Variant | depth (%) | (mJ) | (kPa) | 1/s) |
| 0.1% SP/2.5% CA/5% FR | 74.4 ± 3.1 | 6.86 ± 0.07 | 11.3 ± 2.8 | 132.0 ± 49.6 |
| (F60-2) | | | | |
| 0.1% SP/2.5% CA/10% FR | 78.6 ± 6.6 | 5.18 ± 1.66 | 11.9 ± 1.1 | 188.7 ± 48.1 |
| (F60-3) | | | | |
| 0.1% SP/7.5% CA/5% FR | 70.0 ± 8.4 | 10.50 ± 3.19 | 16.8 ± 1.0 | 62.1 |
| (F60-4) | | | | |
| 0.1% SP/7.5% CA/10% FR | 63.9 ± 5.5 | 10.67 ± 0.28 | 16.0 ± 4.8 | 191.0 ± 90.0 |
| (F60-5) | | | | |
| 0.5% SP/5% CA/7.5% FR | 76.5 ± 2.0 | 10.92 ± 0.76 | 13.6 ± 3.4 | 255.3 ± 32.0 |
| (F60-1) | | | | |

### Example 4: Formulations Containing Drug

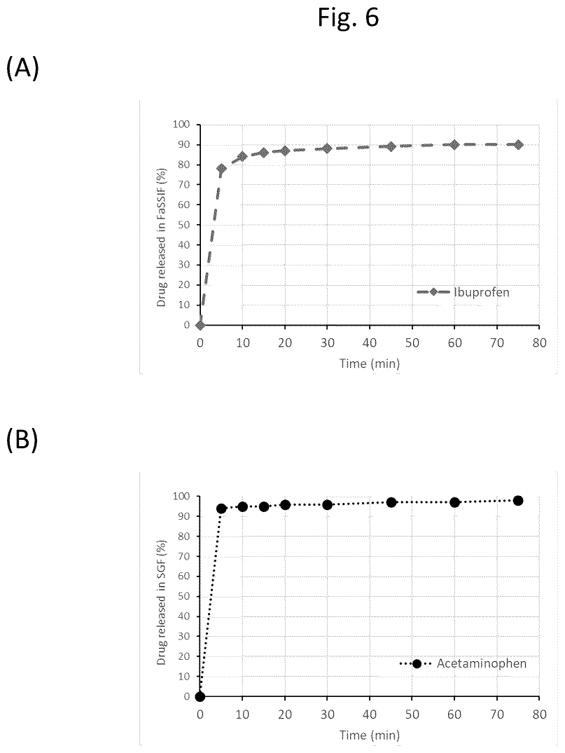

Gel forming tablets may be used as vehicle for mixing after gelling with a drug product. Alternatively, the tablets contain themselves a drug. Formulations combining kappa-carrageenan with polyethylene oxide have shown to release the drug rapidly from the gelled body as shown in FIG. 6 for the drugs acetaminophen and ibuprofen. Table 10 shows the compositions of the formulation including 28.4% of drug substance equivalent to a dose of 100 mg in a tablet with a mass of 352 mg.

TABLE 10
Compositions of drug containing tablet and vehicle tablet

| | Drug containing | | | | |
|---|---|---|---|---|---|
| | tablet | | Vehicle tablet | | |
| | mg per | | mg per | | |
| Ingredients | tablet | % | tablet | % | Function |
| Drug substance* | 100.0 | 28.4 | — | — | Active |
| | | | | | ingredient |
| Polyethylene oxide | 8.75 | 2.5 | 8.75 | 2.5 | Gel former |
| Kappa-carrageenan | 8.75 | 2.5 | 8.75 | 2.5 | Gel former |
| Calcium silicate | 35.0 | 10.0 | 35.0 | 10.0 | Wicking agent |
| Microcrystalline | 87.5 | 24.9 | 87.5 | 24.9 | Filler |
| cellulose | | | | | |
| Mannitol | 110.0 | 31.3 | 210.0 | 59.7 | Filler |
| Sodium stearyl fumarate | 1.75 | 0.5 | 0.5 | 0.5 | lubricant |
| Total: | 352 | 100 | 352 | 100 | — |

*drug substance used: acetaminophen and ibuprofen

The obtained properties of the gelled tablets conform with the targeted characteristics in terms of indentation depth, work of penetration, flow point and shear viscosity as shown in Table 11. The shear viscosity for drug containing tablets at the drug load of 28.4% is below 10 Pa*s but still above 5 Pa*s, which is regarded as the lower limit for semi-solid behavior.

TABLE 11
Results of the characterization of gelling progression after 30 s and
rheological properties after 3 min for systems with polyethylene oxide
and kappa-carrageenan optionally containing 28.4% drug, tablets with
353 mg weight and a solid fraction of 0.75, average ± SD (n = 3):

| | | | Rheometer | |
|---|---|---|---|---|
| | | | | Shear |
| | Texture analyzer | | | viscosity |
| Tablet | Indentation | Penetration Work | Flow point | (Pa*s at 50 |
| Variant | depth (%) | (N*mm) | (kPa) | 1/s) |
| Vehicle | 69.5 ± 2.6 | 10.71 ± 0.54 | 1.4 ± 0.7 | 25.0 ± 22.2 |
| Ibuprofen | 60.0 ± 2.1 | 6.82 ± 0.29 | 9.1 ± 1.2 | 5.7 ± 1.0 |
| Acetaminophen | 77.6 ± 0.2 | 6.71 ± 0.29 | 9.4 ± 0.3 | 7.1 ± 4.0 |

1. A method for preparing a dry solid composition as a precursor for a stable gel body formulation, comprising
a) combining a first gel forming agent and a second gel forming agent with a wicking agent to form a dry solid composition precursor; and
b) adding water to said dry solid composition precursor thereby obtaining a stable gel body formulation.

2. The method of claim 1, wherein the first gel forming agent is kappa-carrageenan.

3. The method of claim 2, wherein the second gel forming agent is polyethylene oxide with an average molecular weight of about 100.000 g/mol, or the second gel forming agent is sodium polyacrylate.

4. The method of claim 3, wherein said kappa-carrageenan and said polyethylene oxide are combined at a ratio (w/w) within the range of 3:1 to 1:3.

5. (canceled)

6. The method of claim 3, wherein said kappa-carrageenan and said sodium polyacrylate are combined at a ratio (w/w) within the range of 2.5:1 to 75:1.

7. The method of claim 1, wherein the first gel forming agent is polyethylene oxide and the second gel forming agent is selected from the group consisting of: pectin and sodium polyacrylate.

8. The method of claim 7, wherein the polyethylene oxide has an average molecular weight of about 100.000 g/mol.

9. The method of claim 7, wherein said polyethylene oxide is combined with pectin at a ratio (w/w) of about 1:1.

10. The method of claim 7, wherein said polyethylene oxide is combined with sodium polyacrylate at a ratio (w/w) of about 10:1.

11. The method of claim 1, wherein the wicking agent is selected from the group consisting of: highly porous calcium silicate, and highly porous magnesium aluminometasilicate.

12. The method of claim 11, wherein the wicking agent is highly porous calcium silicate at a concentration of about 5 to about 10% (w/w) in the dry solid composition.

13. The method of claim 11, wherein the wicking agent is highly porous magnesium aluminometasilicate at a concentration of about 7 to about 8% (w/w) in the dry solid composition.

14. The method of claim 1, wherein the dry solid composition further comprises an active pharmaceutical ingredient and.

15. The method of claim 14, wherein the dry solid composition comprises up to 30% (w/w) of the active pharmaceutical ingredient.

16. The method of claim 15, wherein the active pharmaceutical ingredient is a small molecule drug suitable for oral administration to a patient.

17. (canceled)

18. The method of claim 1, wherein the stable gel body formulation is formed within 30 seconds after the addition of water.

19. The method of claim 1, wherein said stable gel body formulation shows sufficient gel firmness, no disintegration into fractional parts and no solid dry core after a maximum of 3 minutes subsequent to the addition of water.

20. (canceled)

21. A stable gel body formulation prepared by the method of claim 1.

22. A method of preparing a pharmaceutical composition formulation comprising an active pharmaceutical ingredient, comprising
a) combining a first gel forming agent, a second gel forming agent, and an active pharmaceutical ingredient with a wicking agent to form a dry solid composition precursor; and
b) adding water to said dry solid composition precursor thereby obtaining a stable gel body pharmaceutical composition formulation.

23. A method of preparing an oral dosage form for administration of a gel body oral dosage formulation, comprising
a) combining a first gel forming agent, a second gel forming agent, and an active pharmaceutical ingredient with a wicking agent to form a dry solid composition precursor; and
b) adding water to said dry solid composition precursor thereby obtaining a stable gel body oral dosage formulation.
